(19) United States
(12) Patent Application Publication
(10) Pub. No.: US 2015/0105614 A1
(43) Pub. Date: Apr. 16, 2015

(54) Method for endoscopic treatment

(71) Applicant: OLYMPUS MEDICAL SYSTEMS CORP., Tokyo (JP)

(72) Inventors: Makoto IGARASHI, Tokyo (JP); Satoshi TAKEKOSHI, Tokyo (JP)

(73) Assignee: OLYMPUS MEDICAL SYSTEMS CORP., Tokyo (JP)

(21) Appl. No.: 14/054,020

(22) Filed: Oct. 15, 2013

(51) Int. Cl. A61B 17/00; A61N 5/06; A61B 1/05

(52) U.S. Cl. CPC A61B 17/00234; A61B 1/05; A61N 5/0603; A61B 2017/00296
USPC 600/104

(57) ABSTRACT

A method for endoscopic treatment that performs treatment on a subject under an endoscope includes irradiating the subject with white light, switching, after irradiation with the white light, to irradiation of a living tissue of the subject with band-limited light having a predetermined peak wavelength, and administering medicine to the subject after irradiation with the band-limited light.

### BACKGROUND OF THE INVENTION

1. Field of the Invention

The present invention relates to a method for endoscopic treatment.

2. Description of the Related Art

Conventionally, various minimally invasive inspections and operations using an endoscope are performed in the medical field. Operators can insert an endoscope into a body cavity, observe an object, images of which are picked up by an image pickup apparatus provided at a distal end portion of an endoscope insertion portion and perform treatment on a lesioned region as required using a treatment instrument inserted in a treatment instrument channel. Surgery using an endoscope does not require abdominal operation or the like, thus having an advantage of reducing physical burden on a patient.

An endoscope apparatus is configured by including an endoscope, an image processing apparatus connected to the endoscope and an observation monitor. An image pickup device provided at the distal end portion of the endoscope insertion portion picks up an image of the lesioned region and the image is displayed on the monitor. The operator can perform diagnosis or necessary treatment while watching the image displayed on the monitor.

Furthermore, some endoscope apparatuses are able to perform not only normal observation using white light but also special light observation using special light such as infrared light for observation of blood vessels inside.

In the case of an infrared endoscope apparatus, for example, indocyanine green (ICG) having an absorption peak characteristic in near-infrared light in the vicinity of a wavelength of 805 nm is injected as medicine into the blood of the patient. The object is then irradiated with infrared light in the vicinity of a wavelength of 805 nm and in the vicinity of 930 nm from a light source apparatus by time sharing. A signal of an object image picked up by a CCD is inputted to a processor of the infrared endoscope apparatus.

Regarding such an infrared endoscope apparatus, there is a proposal on an apparatus whose processor assigns an image in the vicinity of a wavelength of 805 nm to a green color signal (G), an image in the vicinity of a wavelength of 930 nm to a blue color signal (B), and outputs the signals to a monitor (e.g., see Japanese Patent Application Laid-Open Publication No. 2000-41942). Since the image of infrared light in the vicinity of 805 nm which is more absorbed by the ICG is assigned to the green color, the operator can observe the infrared image with good contrast when the ICG is administered.

For example, in endoscopic submucosal dissection (hereinafter, referred to as “ESD”) using an endoscope to perform incision in a mucous membrane layer where a lesioned region exists and dissect the submucosa or the like, the operator needs to check the position of a relatively thick blood vessel in the mucous membrane so as not to cut the blood vessel by an electric knife or the like, and perform treatment such as incision.

Furthermore, endoscope apparatuses using narrow band light whose center wavelength is 415 nm and 540 nm are also being put to practical use. Using an endoscope apparatus using such narrow band light allows capillary vessels in a shallow layer below the living tissue to be displayed on a monitor.

### SUMMARY OF THE INVENTION

A method for endoscopic treatment according to an aspect of the present invention is a method for endoscopic treatment that performs treatment on a subject under an endoscope, the method including irradiating the subject with white light, switching to irradiation of a living tissue of the subject with narrow band light having a predetermined peak wavelength after irradiation with the white light, and administering medicine to the subject after irradiation with the narrow band light.

### BRIEF DESCRIPTION OF THE DRAWINGS

FIG. 1 is a configuration diagram illustrating a configuration of an endoscope apparatus used for a method for endoscopic treatment according to a first embodiment of the present invention;

FIG. 2 is a diagram illustrating a configuration of a rotating filter 14 according to the first embodiment of the present invention;

FIG. 3 is a diagram illustrating an overall processing flow in narrow band light observation according to the first embodiment of the present invention;

FIG. 4 is a diagram illustrating light absorption characteristics of venous blood according to the first embodiment of the present invention;

FIG. 5 is a flowchart illustrating a flow example of a method for endoscopic treatment of intravascular injection for an esophageal varix in endoscopic injection sclerotherapy according to the first embodiment of the present invention;

FIG. 6 is a diagram illustrating a condition in which a distal end portion of an insertion portion 3a of an endoscope 3 according to the first embodiment of the present invention is brought close to an esophageal variceal region AA in the esophagus ES and the esophageal variceal region AA is included within a range of field of view of the endoscope 3;

FIG. 7 is a diagram illustrating an example of an endoscopic image of the interior of the esophagus ES including the esophageal variceal region AA according to the first embodiment of the present invention;

FIG. 8 is a diagram illustrating a condition in which a needle portion 71a of a local injection device 71 that ejects from the distal end portion of the insertion portion 3a of the endoscope 3 is approaching one of the esophageal variceal regions AA to be treated;

FIG. 9 is a diagram illustrating an example of an endoscopic image in the esophagus ES including the esophageal variceal regions AA in the condition in FIG. 8;

FIG. 10 is a diagram illustrating a case where bleeding has occurred due to local injection according to the first embodiment of the present invention;

FIG. 11 is a diagram illustrating hemostasis treatment on a bleeding point using a high-frequency scalpel 81 according to the first embodiment of the present invention;

FIG. 12 is a flowchart illustrating a flow example of the method for endoscopic treatment of extravascular injection for an esophageal varix in endoscopic injection sclerotherapy according to the first embodiment of the present invention;

FIG. 13 is a diagram illustrating an example of an endoscopic image of the interior of the esophagus ES including an esophageal variceal region AAs deflated by intravascular injection and a periphery thereof according to the first embodiment of the present invention;

FIG. 14 is a diagram illustrating an example of an endoscopic image of the interior of the esophagus ES including a mucous membrane in the periphery of the deflated esophageal variceal region AAs according to the first embodiment of the present invention;

FIG. 15 is a diagram illustrating a case where bleeding has occurred during local injection according to the first embodiment of the present invention;

FIG. 16 is a flowchart illustrating a flow example of a method for endoscopic treatment of medicine administration for ulcerative colitis according to a second embodiment of the present invention;

FIG. 17 is a diagram illustrating a condition in which the distal end portion of the insertion portion 3a of the endoscope 3 according to the second embodiment of the present invention is brought close to an ulcerative colitis region (hereinafter, referred to as “UC region”) in a large-intestinal lumen CI of the large intestine CO and the UC region is included in a range of field of view of the endoscope 3;

FIG. 18 is a diagram illustrating an example of an endoscopic image of the interior of the large-intestinal lumen CI including the UC region in a normal light observation mode according to the second embodiment of the present invention;

FIG. 19 is a diagram illustrating an example of an endoscopic image of the interior of the large-intestinal lumen CI including the UC region in a narrow band observation mode according to the second embodiment of the present invention;

FIG. 20 is a diagram illustrating a condition in which a needle portion 71a of a local injection device 71 from the distal end portion of the insertion portion 3a of the endoscope 3 according to the second embodiment of the present invention is approaching the UC region to be treated;

FIG. 21 is a diagram illustrating an example of an endoscopic image of the interior of the large-intestinal lumen CI including the UC region in the condition in FIG. 20;

FIG. 22 is a diagram illustrating a relationship between wavelength and intensity of band-limited light including narrow band light having one predetermined peak wavelength and having a broad range;

FIG. 23 is a diagram illustrating a relationship between wavelength and intensity of band-limited light including narrow band light having two predetermined peak wavelengths and having a broad range; and

FIG. 24 is a diagram illustrating a relationship between wavelength and intensity of band-limited light including narrow band light having one predetermined peak wavelength and one ray of wide band light.

### DETAILED DESCRIPTION OF THE PREFERRED EMBODIMENTS

Hereinafter, embodiments of the present invention will be described with reference to the accompanying drawings.

### First Embodiment

### 1. Configuration of Endoscope Apparatus

Hereinafter, an embodiment of the present invention will be described with reference to the accompanying drawings.

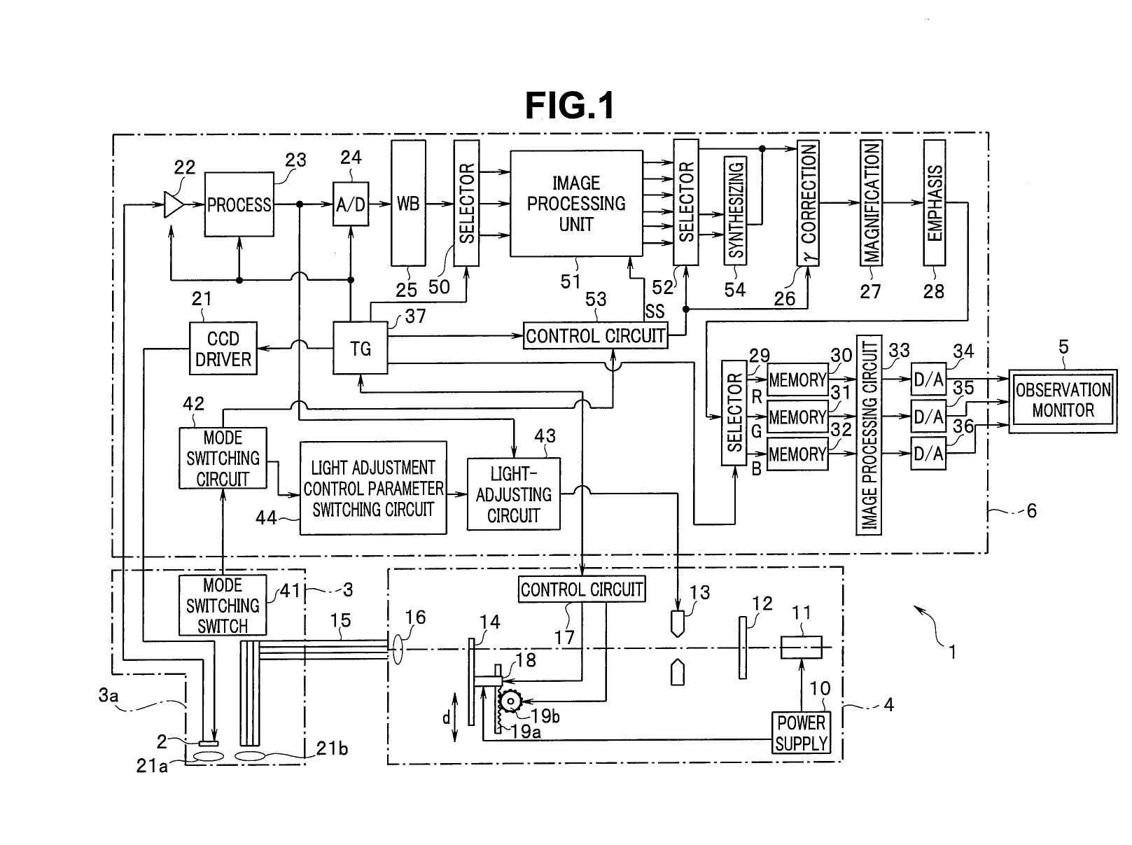

FIG. 1 is a configuration diagram illustrating a configuration of an endoscope apparatus used for a method for endoscopic treatment according to the present embodiment.

As shown in FIG. 1, an endoscope apparatus 1 of the present embodiment is constructed of an electronic endoscope 3 having a CCD 2 which is an image pickup device as a physiological image information acquiring section inserted into a body cavity to pick up an image of a tissue in the body cavity, a light source apparatus 4 that supplies illuminating light to the electronic endoscope (hereinafter, also simply referred to as “endoscope”) 3, and a video processor 6 that applies signal processing to an image pickup signal from the CCD 2 of the electronic endoscope 3 and displays an endoscopic image on an observation monitor 5. The endoscope apparatus 1 includes two modes: a normal light observation mode and a narrow band light observation mode. Note that in the following description, since the normal light observation mode of the endoscope apparatus 1 is the same as a conventional normal light observation mode, description of the configuration of the normal light observation mode is omitted and the narrow band light observation mode will be mainly described.

The endoscope 3 includes an elongated insertion portion 3a and a bending portion (not shown) is provided on a distal end side of the insertion portion 3a. The insertion portion 3a includes a distal end rigid portion on a distal end side of the bending portion and the distal end rigid portion is provided with the CCD 2. The CCD 2 constitutes an image pickup section that receives return light of illuminating light radiated onto a subject and picks up an image of the subject. A forceps channel is provided in the insertion portion as a treatment instrument insertion channel.

The light source apparatus 4 as an illumination section is configured by including a xenon lamp 11 that emits illuminating light (white light), a heat radiation cut filter 12 that cuts heat radiation of the white light, a diaphragm apparatus 13 that controls light quantity of the white light via the heat radiation cut filter 12, a rotating filter 14 as a band-limiting section that transforms the illuminating light into frame-sequential light, a condensing lens 16 that condenses the frame-sequential light onto a plane of incidence of a light guide 15 provided in the endoscope 3 via the rotating filter 14 and a control circuit 17 that controls the rotation and position of the rotating filter 14. The xenon lamp 11, the rotating filter 14 and the light guide 15 constitute an irradiation section that irradiates the subject with illuminating light.

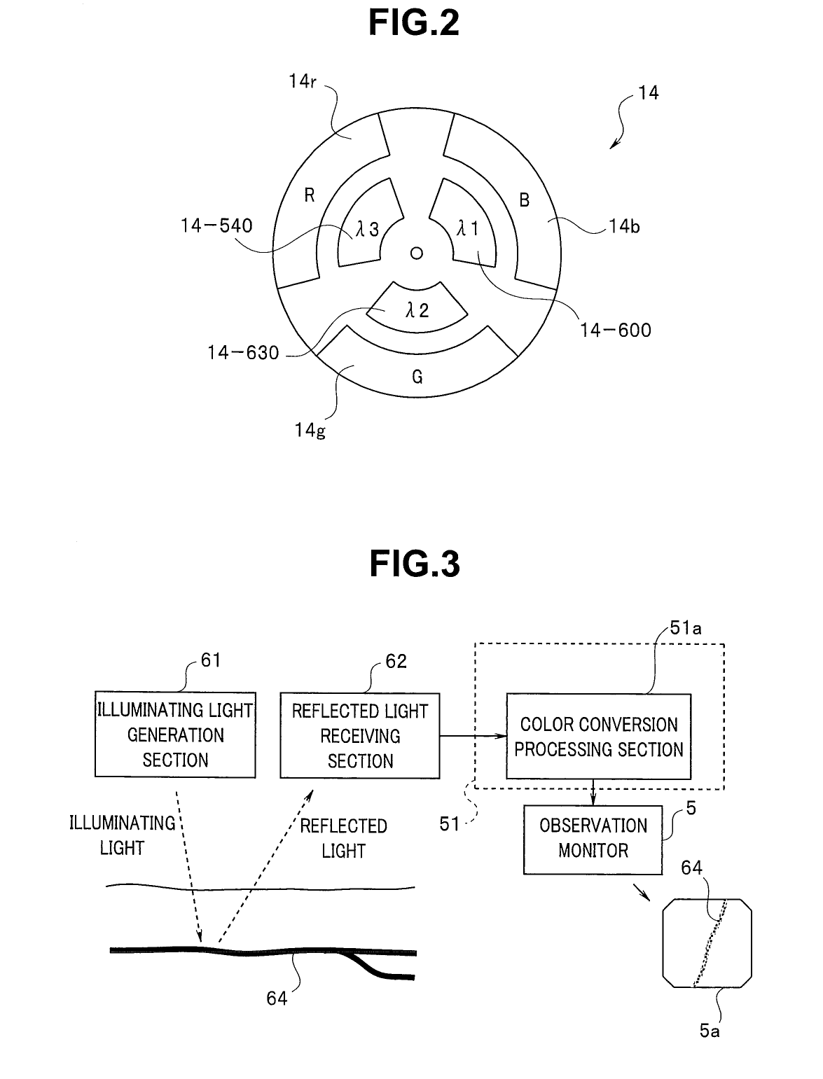

FIG. 2 is a diagram illustrating a configuration of the rotating filter 14. The rotating filter 14 is a filter that allows light from the xenon lamp 11 which is a light source to pass therethrough. The rotating filter 14 as a wavelength band-limiting section is configured into a disk shape as shown in FIG. 2, has a structure whose center is the axis of rotation and includes two filter groups. An R (red) filter section 14r, a G (green) filter section 14g and a B (blue) filter section 14b constituting a filter set to output frame-sequential light having spectral characteristics for normal light observation are arranged along a circumferential direction on an outer circumferential side of the rotating filter 14 as a first filter group.

Three filters 14-600, 14-630 and 14-540 that allow three light beams of predetermined narrow band wavelengths to pass therethrough are arranged along a circumferential direction on an inner circumferential side of the rotating filter 14 as a second filter group.

The filter 14-600 is configured so as to allow narrow band light in the vicinity of a wavelength of 600 nm (λ1) to pass therethrough as band-limited light. The filter 14-630 is configured so as to allow narrow band light in the vicinity of a wavelength of 630 nm (λ2) to pass therethrough as band-limited light. The filter 14-540 is configured so as to allow narrow band light in the vicinity of a wavelength of 540 nm (λ3) to pass therethrough as band-limited light.

Here, the term “vicinity” in the case of in the vicinity of a wavelength of 600 nm means narrow band light having a center wavelength of 600 nm and a width with a range of distribution of, for example, 20 nm centered on the wavelength of 600 nm (that is, from wavelength 590 nm to 610 nm around the wavelength of 600 nm). The same applies to the other wavelengths: wavelength 630 nm and wavelength 540 nm which will be described later.

The rotating filter 14 is arranged on an optical path from the xenon lamp 11 which is an illuminating light emitting section to an image pickup surface of the CCD 2 to place a limit on at least one (three here) of a plurality of wavelength bands of the illuminating light in each mode so as to narrow the wavelength bands.

The control circuit 17 then controls a motor 18 to rotate the rotating filter 14 and controls the rotation of the rotating filter 14.

A rack 19a is connected to the motor 18, a motor (not shown) is connected to a pinion 19b, and the rack 19a is threadably mounted on the pinion 19b. The control circuit 17 controls the rotation of the motor connected to the pinion 19b, and can thereby move the rotating filter 14 in a direction shown by an arrow d. Thus, the control circuit 17 controls the motor connected to the pinion 19b so as to place the first filter group in a normal light observation mode and the second filter group in a narrow band light observation mode on an optical path in accordance with a mode switching operation by a user, which will be described later.

Note that power is supplied to the xenon lamp 11, the diaphragm apparatus 13, the rotating filter motor 18 and the motor (not shown) connected to the pinion 19b from a power supply section 10.

Thus, the light source apparatus 4 constitutes an illumination section that irradiates the subject with at least one or more illuminating light beams (three band-limited light beams, here) having predetermined wavelength bands in the narrow band light observation mode. Here, one of the three illuminating light beams is a narrow band light beam to clearly display a blood vessel in a depth of 1 to 2 mm from a surface layer portion of a mucous membrane, and the remaining two are a narrow band light beam to display a deeper blood vessel and a narrow band light beam to display a capillary vessel in a range near the surface layer portion. For this reason, the light source apparatus 4 is an illumination apparatus that radiates at least one or more illuminating light beams via the band-limiting section that limits light to a first wavelength band (which will be described later) in the narrow band light observation mode.

The video processor 6 is configured by including a CCD drive circuit 21 which is a CCD driver, an amplifier 22, a process circuit 23, an A/D converter 24, a white balance circuit (hereinafter, referred to as “WB”) 25, a selector 50, an image processing unit 51, a selector 52, a γ correction circuit 26, a magnification circuit 27, an emphasis circuit 28, a selector 29, synchronization memories 30, 31 and 32, an image processing circuit 33, D/A converters 34,35 and 36, a timing generator (hereinafter, referred to as “TG”) 37, a mode switching circuit 42, a light-adjusting circuit 43, a light adjustment control parameter switching circuit 44, a control circuit 53, and a synthesizing circuit 54 as a display image generation section.

The CCD drive circuit 21 is intended to drive the CCD 2 provided in the endoscope 3 and output a frame-sequential image pickup signal synchronized with the rotation of the rotating filter 14 to the CCD 2. Furthermore, the amplifier 22 is intended to amplify a frame-sequential image pickup signal obtained by the CCD 2 picking up an image of a tissue in the body cavity via an objective optical system 21a provided at a distal end of the endoscope 3. Furthermore, an illumination optical system 21b is provided on a distal end side of the light guide 15.

Note that polarizing plates in a crossed Nichol state may be arranged on a front surface of the CCD 2 which is an image pickup device and on a front surface of the light guide 15 respectively. The two polarizing plates in a crossed Nichol state allow the CCD 2 to pick up an image of only light from a mucous membrane depth without receiving reflected light from the mucous membrane surface.

The process circuit 23 performs correlated double sampling and noise cancellation or the like on the frame-sequential image pickup signal via the amplifier 22. The A/D converter 24 converts the frame-sequential image pickup signal that has passed through the process circuit 23 to a digital frame-sequential image signal.

The WB 25 performs gain adjustment and white balance processing on the frame-sequential image signal digitized by the A/D converter 24 so that the brightness of an R signal of the image signal is equivalent to the brightness of a B signal of the image signal with reference to a G signal of the image signal, for example.

Note that the white balance adjustment in the WB 25 is performed with reference to the luminance of return light of narrow band light in the vicinity of a wavelength of 600 nm.

The selector 50 assigns and outputs the frame-sequential image signal from the WB 25 into respective sections in the image processing unit 51.

The image processing unit 51 is an image signal processing section that converts an RGB image signal for normal light observation or three image signals for narrow band light observation from the selector 50 to image signals for display. The image processing unit 51 outputs image signals in a normal light observation mode and a narrow band light observation mode to the selector 52 according to a selection signal SS from the control circuit 53 based on a mode signal.

The selector 52 sequentially outputs the image signal for normal light observation and the image signal for narrow band light observation from the image processing unit 51 to the γ correction circuit 26 and the synthesizing circuit 54.

The γ correction circuit 26 applies γ correction processing to the frame-sequential image signal from the selector 52 or the synthesizing circuit 54. The magnification circuit 27 performs magnification processing on the frame-sequential image signal subjected to the γ correction processing in the γ correction circuit 26. The emphasis circuit 28 applies contour emphasis processing to the frame-sequential image signal subjected to the magnification processing in the magnification circuit 27. The selector 29 and the synchronization memories 30, 31 and 32 are intended to synchronize the frame-sequential image signals from the emphasis circuit 28.

The image processing circuit 33 reads the respective frame-sequential image signals stored in the synchronization memories 30, 31 and 32 and performs moving image color drift correction processing or the like. The D/A converters 34, 35 and 36 convert the image signals from the image processing circuit 33 to RGB analog video signals and outputs the signals to the observation monitor 5. The TG 37 receives a synchronization signal which is synchronized with the rotation of the rotating filter 14 from the control circuit 17 of the light source apparatus 4 and outputs various timing signals to the respective circuits in the video processor 6.

Furthermore, the endoscope 3 is provided with a mode switching switch 41 for switching between the normal light observation mode and the narrow band light observation mode, and the output of the mode switching switch 41 is designed to be outputted to the mode switching circuit 42 in the video processor 6. The mode switching circuit 42 of the video processor 6 is designed to output a control signal to the light adjustment control parameter switching circuit 44 and the control circuit 53. The light-adjusting circuit 43 is designed to control the diaphragm apparatus 13 of the light source apparatus 4 based on a light adjustment control parameter from the light adjustment control parameter switching circuit 44 and the image pickup signal after passing through the process circuit 23 to perform appropriate brightness control.

The respective circuits in the video processor 6 perform predetermined processing in accordance with a specified mode. Those circuits perform processing in accordance with the normal light observation mode and the narrow band light observation mode respectively, and the observation monitor 5 displays an image for normal light observation or an image for narrow band light observation. As will be described later, in the narrow band light observation mode, the observation monitor 5 displays an image based on an image signal of a relatively thick blood vessel having a diameter on the order of 1 to 2 mm at a depth of the mucous membrane on the order of 1 to 2 mm from the surface layer portion of the mucous membrane.

### 2. Overall Processing Flow of Narrow Band Light Observation

Next, an overall approximate flow of narrow band light observation according to the present embodiment will be described briefly.

FIG. 3 is a diagram illustrating an overall processing flow in narrow band light observation according to the present embodiment.

The operator inserts the insertion portion of the endoscope into the body cavity and places the distal end portion of the endoscope insertion portion in the vicinity of a lesioned region in a normal light observation mode. To observe a relatively thick blood vessel of, for example, 1 to 2 mm in diameter, in the depth running through the muscularis propria from the submucosa, the operator operates the mode switching switch 41 to switch the observation mode of the endoscope apparatus 1 to the narrow band light observation mode.

In the narrow band light observation mode, the control circuit 17 of the endoscope apparatus 1 controls the motor connected to the pinion 19b to move the position of the rotating filter 14 so as to emit light that has passed through the second filter group from the light source apparatus 4. The control circuit 53 also controls the various circuits in the video processor 6 so as to perform image processing for observation using a narrow band wavelength.

As shown in FIG. 3, in the narrow band light observation mode, illuminating light having a narrow band wavelength is emitted from an illuminating light generation section 61 from the distal end portion of the insertion portion of the endoscope 3, and after passing through the mucous membrane layer, radiated onto the blood vessel 64 running through the submucosa and the muscularis propria. Here, the illuminating light generation section 61 is configured by including the light source apparatus 4, the rotating filter 14 and the light guide 15 or the like, and emits illuminating light from the distal end of the endoscope insertion portion. As the rotating filter 14 rotates, narrow band light in the vicinity of a wavelength of 600 nm, narrow band light in the vicinity of a wavelength of 630 nm and narrow band light in the vicinity of a wavelength of 540 nm are consecutively and sequentially emitted from the light source apparatus 4 as band-limited light beams and radiated onto the object.

Reflected light beams of the narrow band light in the vicinity of a wavelength of 600 nm, the narrow band light in the vicinity of a wavelength of 630 nm and the narrow band light in the vicinity of a wavelength of 540 nm are respectively received by a reflected light receiving section 62 which is the CCD 2. The CCD 2 outputs image pickup signals of the respective reflected light beams and supplies the image pickup signals to the selector 50 via the amplifier 22 or the like. The selector 50 maintains a first image signal P1 in the vicinity of a wavelength of 600 nm, a second image signal P2 in the vicinity of a wavelength of 630 nm and a third image signal P3 in the vicinity of a wavelength of 540 nm in accordance with predetermined timing from the TG 37 and supplies the image signals to the image processing unit 51. The image processing unit 51 includes a color conversion processing section 51a for the narrow band light observation mode.

The operator can set the endoscope apparatus 1 to the narrow band light observation mode to cause the relatively thick blood vessel in the depth of the mucous membrane to be displayed on a screen 5a of the observation monitor 5 as shown in FIG. 3 in, for example, red color or magenta color with relatively high contrast.

Furthermore, the operator can also set the endoscope apparatus 1 to the narrow band light observation mode to cause not only the blood vessel below the surface of a living tissue but also a bleeding point at which bleeding has occurred to be drawn on the observation monitor 5. This is because even when bleeding occurs from the bleeding point on the mucous membrane surface of the mucous membrane and the mucous membrane surface is covered with the blood, when the blood is observed in the narrow band light observation mode, narrow band light in the vicinity of a wavelength of 600 nm passes through the blood and the blood running from the bleeding point on the mucous membrane surface is displayed on the observation monitor 5. Since a variation in a density (that is, concentration) of the blood flowing from the bleeding point or a variation in the thickness of the blood layer is high in the vicinity of the bleeding point, the flow of the blood flowing from the bleeding point is visualized so that the operator can visually recognize the blood flow, identify the bleeding point below the blood and the operator can speedily apply hemostasis treatment to the bleeding point.

Therefore, the color conversion processing section 51a of the image processing unit 51 in FIG. 1 assigns the respective image signals to respective channels of RGB of the observation monitor 5 and supplies the image signals to the selector 52. As a result, the relatively thick blood vessel 64 in the depth of the mucous membrane and the bleeding point at which bleeding has occurred are displayed on the screen 5a of the observation monitor 5 with high contrast.

For example, in order for the color conversion processing section 51a to display a blood vessel 64 in the depth with high contrast using narrow band light NL1 in the vicinity of a wavelength of 600 nm, the color conversion processing section 51a assigns the first image signal P1 (λ1), the second image signal P2 (λ2) and the third image signal P3 (λ3) to the G, R and B channels respectively.

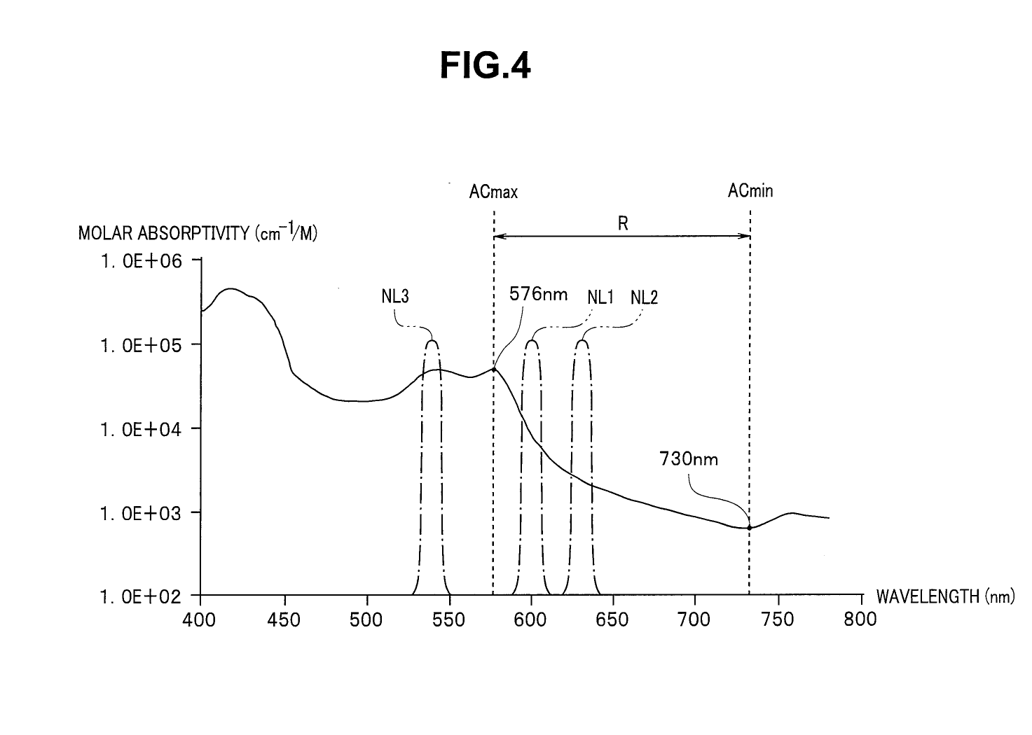

Here, light absorption characteristics of venous blood will be described. FIG. 4 is a diagram illustrating light absorption characteristics of venous blood. The vertical axis in FIG. 4 shows molar absorptivity (cm⁻¹/M) and the horizontal axis shows wavelength. Note that although three narrow band illuminating light beams are affected by scattering characteristics of the living tissue itself, the scattering characteristics of the living tissue itself monotonously decrease as the wavelength increases, and therefore FIG. 4 will be described as the absorption characteristics of the living tissue.

Generally, the venous blood contains oxygenated hemoglobin (HbO₂) and reduced hemoglobin (Hb) (hereinafter, both will be simply jointly referred to as “hemoglobin”) at a proportion of 60:40. Light is absorbed by hemoglobin, but the absorption coefficient thereof varies from one wavelength of light to another. FIG. 4 shows light absorption characteristics of venous blood for each wavelength from 400 nm to approximately 800 nm, and the absorptivity shows a maximum value at a point of wavelength of approximately 576 nm and a minimum value at a point of wavelength of 730 nm in a range from 550 nm to 750 nm.

In the narrow band light observation mode, three narrow band light beams are radiated and their respective return light beams are received by the CCD 2.

The narrow band light in the vicinity of a wavelength of 600 nm (hereinafter referred to as “first narrow band light NL1”) is light in a wavelength band within a wavelength band R from a maximum value ACmax (here, absorptivity at a wavelength of 576 nm) to a minimum value ACmin (here, absorptivity at a wavelength of 730 nm) of absorption characteristics of hemoglobin.

The narrow band light in the vicinity of a wavelength of 630 nm (hereinafter, also referred to as “second narrow band light NL2”) is also light within the wavelength band R from the maximum value ACmax to the minimum value ACmin of absorption characteristics of hemoglobin, but it is light in a wavelength band having a longer wavelength than the first narrow band light NL1, lower absorptivity and with suppressed scattering characteristics of a living tissue. The suppressed scattering characteristics mean that the scattering coefficient decreases toward the long wavelength side.

That is, the light source apparatus 4 radiates first illuminating light NL1 having a peak wavelength in spectral characteristics between the wavelength band including the maximum value ACmax and the wavelength band including the minimum value ACmin in the absorption characteristics of the living tissue.

Furthermore, the light source apparatus 4 also radiates second illuminating light NL2 having lower absorption characteristic values than the image signal P1 resulting from the first illuminating light NL1 and having a peak wavelength in spectral characteristics with suppressed scattering characteristics of the living tissue.

Moreover, the light source apparatus 4 also radiates narrow band light in the vicinity of a wavelength of 540 nm (hereinafter, referred to as “third narrow band light NL3”). The third narrow band light NL3 is light in a wavelength band other than the wavelength band R from the maximum value ACmax to the minimum value ACmin in the absorption characteristics of hemoglobin and is illuminating light transmittable by a predetermined distance from the surface layer portion of the mucous membrane surface of the subject.

The CCD 2 outputs image pickup signals of the respective images of three narrow band light beams. Thus, each image includes a plurality of pixel signals based on respective return light beams of the first, second and third narrow band light beams NL1, NL2 and NL3.

The first narrow band light NL1 and the second narrow band light NL2 repeat multiple scattering processes in the living tissue respectively, and are consequently emitted from the mucous membrane surface as return light. The first narrow band light NL1 and the second narrow band light NL2 have their respective mean free paths. The mean free path of the first narrow band light NL1 is shorter than the mean free path of the second narrow band light NL2.

Thus, the first narrow band light NL1 in the vicinity of a wavelength of 600 nm (λ1) reaches the vicinity of the blood vessel 64 and the second narrow band light NL2 in the vicinity of a wavelength of 630 nm (λ2) reaches a position slightly deeper than the blood vessel 64. Using this first narrow band light NL1 thereby makes it possible to display a relatively thick blood vessel having a diameter of 1 to 2 mm and a bleeding point at which bleeding has occurred, located in a relatively deep part, 1 to 2 mm below the surface layer of the mucous membrane of the living body.

The second narrow band light NL2 in the vicinity of a wavelength of 630 nm (λ2) also makes it possible to display a thicker blood vessel and a bleeding point at which bleeding has occurred, located in a deeper part.

Here, although the narrow band light NL1 or NL2 is light in the aforementioned wavelength band, the range of light in which the relatively thick blood vessel can be displayed with high contrast is from 585 nm which is the minimum wavelength to 630 nm which is the maximum wavelength.

The endoscope apparatus 1 radiates the above-described narrow band light, and can thereby cause the observation monitor 5 to display the blood vessel in the living tissue and the bleeding point at which bleeding has occurred.

As described above, the operator can use the aforementioned endoscope apparatus 1 to irradiate the subject with white light, irradiate the subject with narrow band light having a predetermined peak wavelength or switch from irradiation with white light to irradiation with narrow band light. The narrow band light is light in a red band of a visible range and including narrow band light having a peak wavelength in spectral characteristics between the wavelength band including a maximum value and the wavelength band including a minimum value in the hemoglobin light absorption characteristics of the living tissue of the subject.

### 3. Flow of Endoscopic Treatment of Endoscopic Injection Sclerotherapy (EIS) for Esophageal Varix

Next, an example of a method for endoscopic treatment that performs treatment on a subject under an endoscope according to the present embodiment will be described.

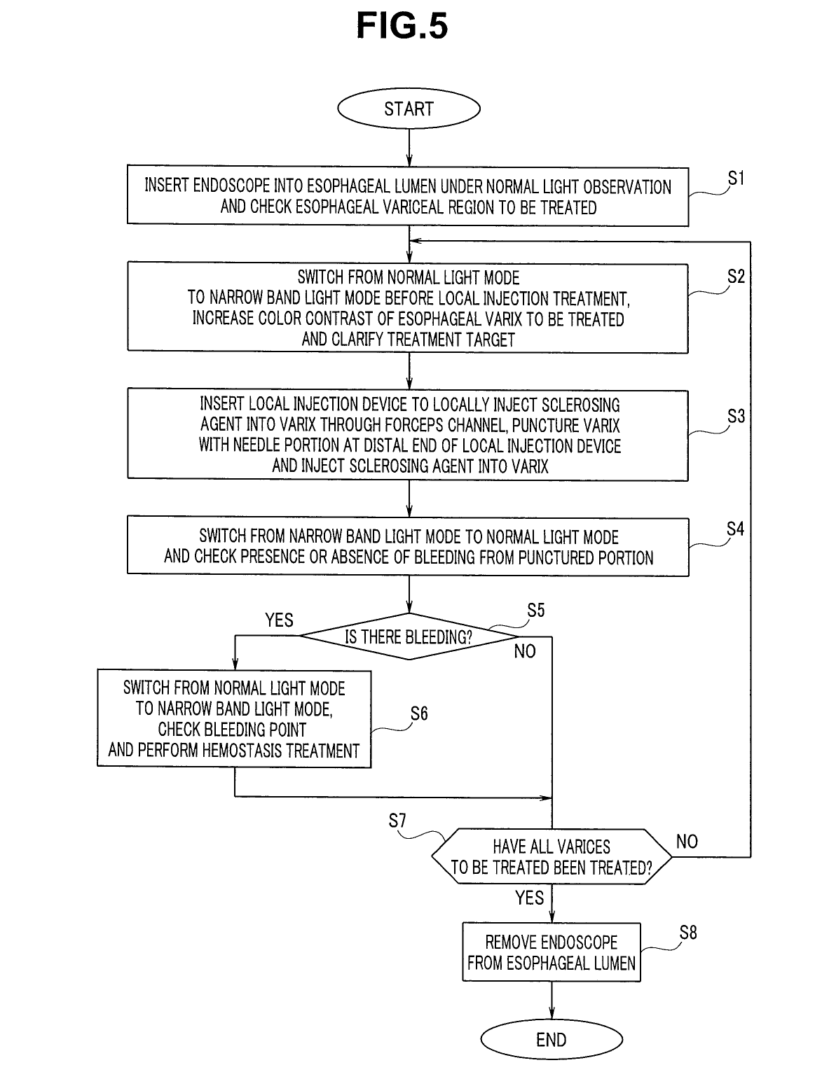

FIG. 5 is a flowchart illustrating a flow example of a method for endoscopic treatment of intravascular injection for an esophageal varix in endoscopic injection sclerotherapy (hereinafter, referred to as “EIS”). EIS includes medicine administration (intravascular injection here) to the interior of a varix (that is, into a blood vessel) and medicine administration (extravascular injection here) to the periphery of the varix (that is, outside a blood vessel).

A method for endoscopic treatment of intravascular injection for an esophageal varix according to the present embodiment will be described first and then treatment of extravascular injection for the esophageal varix will be described.

### 3.1 Method for Endoscopic Treatment of Intravascular Injection

### [Check of Esophageal Variceal Region]

The operator inserts the endoscope 3 into the esophageal lumen under normal light observation and checks the esophageal variceal region to be treated (S1). That is, the subject is irradiated with white light first.

More specifically, the operator sets the operating mode of the endoscope apparatus 1 to a normal light observation mode, inserts the insertion portion 3a from the mouth of the subject, watches an image on the observation monitor 5, causes the distal end portion of the insertion portion 3a to approach the vicinity of the esophageal varix while operating the bending portion and checks the esophageal variceal region to be treated. Note that there may be a case where the esophageal varix cannot be visually recognized.

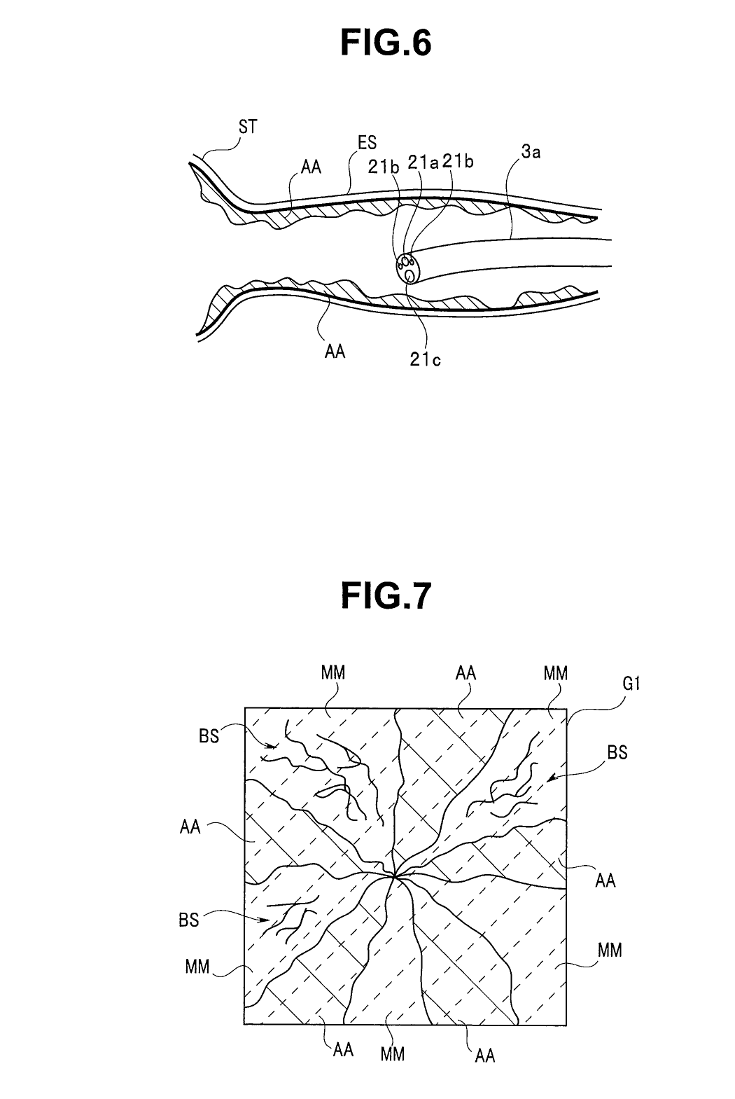

FIG. 6 and FIG. 7 are diagrams illustrating checking of the esophageal variceal region. FIG. 6 is a diagram illustrating a condition in which the distal end portion of the insertion portion 3a of the endoscope 3 is brought close to an esophageal variceal region AA in the esophagus ES and the esophageal variceal region AA is included within a range of field of view of the endoscope 3. An objective optical system 21a provided in the aforementioned observation window, an illumination optical system 21b provided in an illuminating window and an opening 21c of a forceps channel (not shown) are provided at the distal end portion of the insertion portion 3a of the endoscope 3.

As shown in FIG. 6, a plurality of esophageal variceal regions AA exist in the esophagus ES on the upstream side of the stomach ST. When the operator brings the distal end portion of the insertion portion 3a of the endoscope 3 close to the plurality of esophageal variceal regions AA, an endoscopic image of the interior of the esophagus ES as shown in FIG. 7 is displayed on the observation monitor 5.

FIG. 7 is a diagram illustrating an example of an endoscopic image of the interior of the esophagus ES including the esophageal variceal regions AA. As an endoscopic image G1, the plurality of esophageal variceal regions AA formed on the inner wall of the esophagus ES and a plurality of esophageal mucous membrane regions MM are displayed on the screen 5a of the observation monitor 5. Surface-layer blood vessels BS appear in some esophageal mucous membrane regions MM.

Since FIG. 7 is an endoscopic image under normal light observation, the esophageal mucous membranes MM, the esophageal variceal regions AA and the surface-layer blood vessels BS are all displayed in red and the contrast of the esophageal variceal regions AA with respect to the surrounding esophageal mucous membranes MM is low. In FIG. 7, dotted diagonal lines are placed over the entire endoscopic image G1, indicating that the esophageal variceal regions AA have low contrast with respect to the surrounding esophageal mucous membranes MM.

### [Switching to Narrow Band Observation Mode]

Returning to FIG. 5, the operator switches the operating mode of the endoscope apparatus 1 from the normal light observation mode to the narrow band light observation mode before applying local injection to the plurality of esophageal variceal regions AA respectively, thereby increases the color contrast of the esophageal varix to be treated and clarifies the treatment target (S2). That is, after irradiation with white light, switchover is made to irradiation of a living tissue of the subject with narrow band light having a predetermined peak wavelength.

This is because by switching the observation mode from the normal light observation mode to the narrow band light observation mode before local injection treatment, it is possible to display the esophageal variceal regions which are relatively thick blood vessels in the depth of the mucous membrane on the observation monitor 5 with high contrast and in a visually recognizable manner.

### [Local Injection]

Next, the operator inserts a local injection device to locally inject a sclerosing agent into the esophageal varix through the forceps channel, punctures the esophageal varix with the needle portion at the distal end of the local injection device and injects the sclerosing agent into the esophageal varix (S3). That is, after irradiation with narrow band light, medicine is administered to the subject.

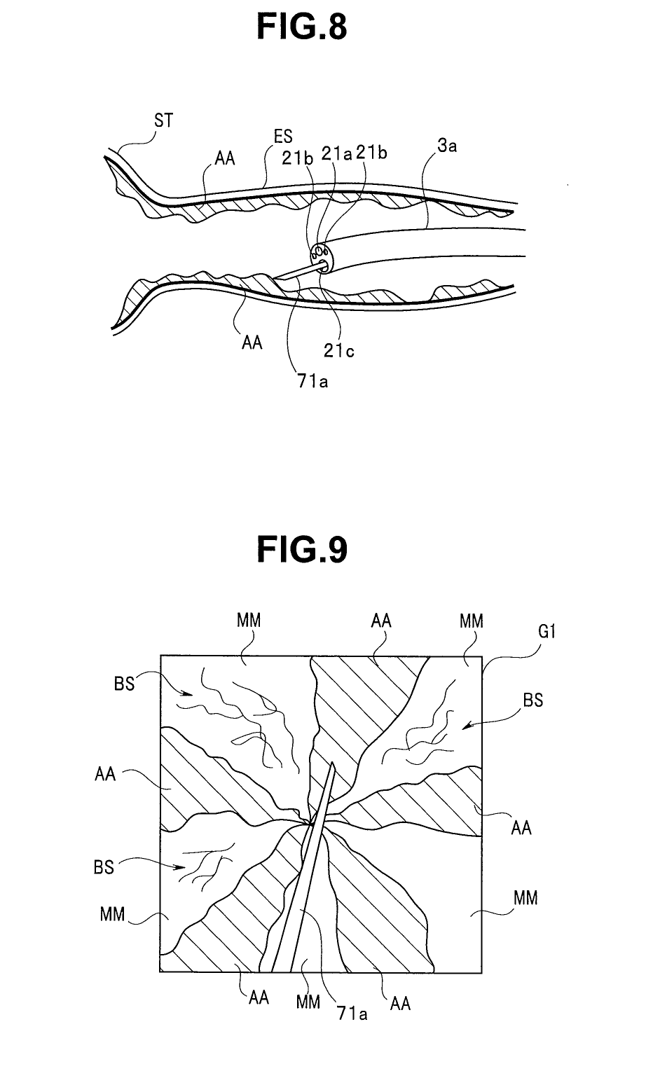

FIG. 8 and FIG. 9 are diagrams illustrating local injection into the esophageal variceal region. FIG. 8 is a diagram illustrating a condition in which a needle portion 71a of the local injection device 71 that ejects from the distal end portion of the insertion portion 3a of the endoscope 3 is approaching one of the esophageal variceal regions AA to be treated. FIG. 9 is a diagram illustrating an example of an endoscopic image of the interior of the esophagus ES including the esophageal variceal regions AA in the condition in FIG. 8. As shown in FIG. 8, medicine is administered by locally injecting the sclerosing agent into the varices of the subject via the local injection needle.

Since FIG. 9 is an endoscopic image under narrow band light observation, the esophageal mucous membranes MM are displayed in yellow, the esophageal variceal regions AA are displayed in dark red and the surface-layer blood vessels BS are displayed in yellow or orange, the esophageal variceal regions AA are displayed with high contrast with respect to the surrounding esophageal mucous membranes MM. The dotted diagonal lines shown in FIG. 7 are not placed over the entire endoscopic image G1 in FIG. 9, indicating that the esophageal variceal regions AA in FIG. 9 have higher contrast with respect to the surrounding esophageal mucous membranes MM under narrow band light observation.

An example of the sclerosing agent is Oldamin (monoethanolamine oleate). After the local injection, the varices are caused by the sclerosing agent to solidify and disappear. Since the endoscopic image G1 in the narrow band light observation mode shows a situation in which the color contrast of blood vessels of the varices is reduced, the operator can determine result of the treatment by the medicine in real time while watching the observation monitor 5. It is difficult to determine results of the treatment of the varices in real time in the normal light observation mode.

### [Checking Presence or Absence of Bleeding]

Next, the operator switches the observation mode from the narrow band light observation mode to the normal light observation mode and checks the presence or absence of bleeding from the portion punctured with the needle portion 71a (S4).

When bleeding is detected (S5: YES) as a result of determining the presence or absence of bleeding (S5), the operator performs hemostasis treatment (S6).

### [Hemostasis Treatment]

The operator switches the observation mode from the normal light observation mode to the narrow band light observation mode, checks the bleeding point and performs hemostasis treatment (S6).

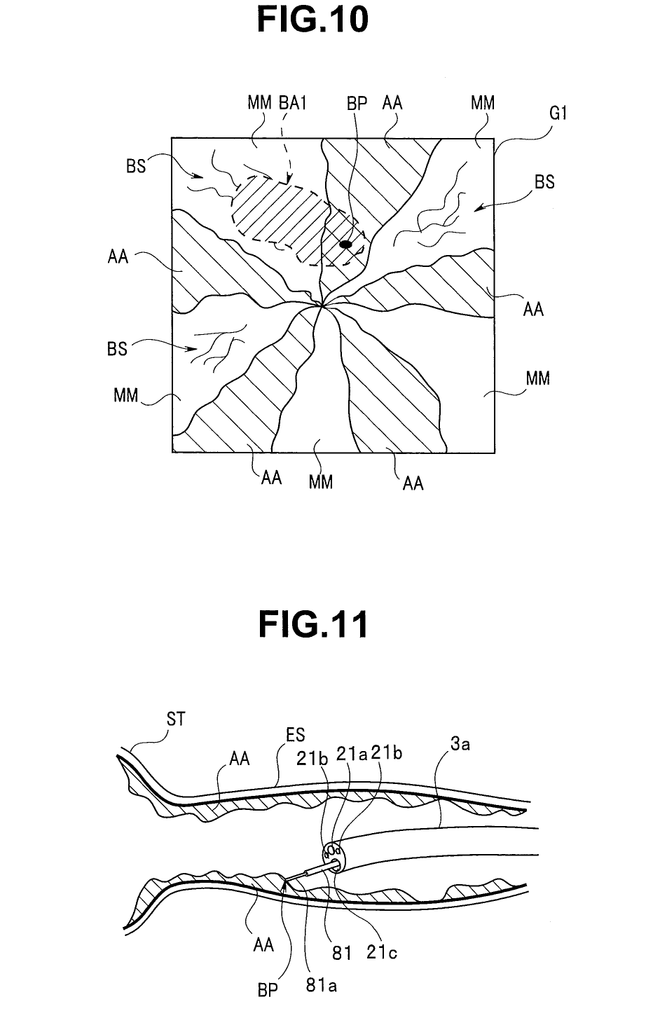

FIG. 10 is a diagram illustrating a case where bleeding has occurred due to local injection. After bleeding is detected, when the observation mode is switched to the narrow band light observation mode, the operator can visually recognize the bleeding point BP on the observation monitor 5 in the narrow band light observation mode as shown in FIG. 10. In the narrow band light observation mode, since a bleeding region BA1 shown by diagonal lines is displayed in yellow to orange, the bleeding point BP in the bleeding region BA1 is displayed in yellow, the operator can identify the bleeding point BP.

Upon successfully identifying the position of the bleeding point BP, the operator can perform astriction treatment using a balloon provided in the insertion portion 3a or effectively perform hemostasis treatment by inserting the high-frequency scalpel 81 or hemostasis forceps through the forceps channel.

FIG. 11 is a diagram illustrating hemostasis treatment on the bleeding point using the high-frequency scalpel 81. As shown in FIG. 11, in the case of the high-frequency scalpel 81, hemostasis treatment is performed by causing a distal end portion 81a of the high-frequency scalpel 81 inserted through the forceps channel to contact the bleeding point BP and passing a high-frequency current therethrough. In the case of the hemostasis forceps, hemostasis treatment is performed by grasping the bleeding point BP with the surface of the grasping portion at the distal end of the hemostasis forceps and passing a high-frequency current therethrough.

After the hemostasis treatment, the operator switches the observation mode from the narrow band light observation mode to the normal light observation mode, thereby switches from irradiation with narrow band light to irradiation with white light, and confirms whether or not the hemostasis treatment has been completed.

After confirming that the hemostasis treatment has been completed, the operator determines whether or not all the esophageal varices to be treated have been treated (S7), and if there still remain esophageal varices to be treated (S7: NO), the operator returns to S2 and repeats the aforementioned treatments in S2 to S6 on the other esophageal variceal regions AA.

When all the esophageal varices to be treated have been treated and there remain no esophageal varices to be treated (S7: YES), the operator removes the endoscope 3 from the esophageal lumen (S8).

In treatment of esophageal varices in general, the esophageal varices can be reduced in size by repeating intravascular injections in the beginning of the treatment.

3.2 Method for Endoscopic Treatment with Extravascular Injection

Next, endoscopic injection sclerotherapy using extravascular injection into an esophageal varix will be described.

After the esophageal varix is deflated by intravascular injection or after the esophageal varix disappears, prevention of recurrence of an esophageal varix can be expected by performing extravascular injection to induce rupture of veins in the periphery of the esophageal varix and fibrillization of the mucous membrane.

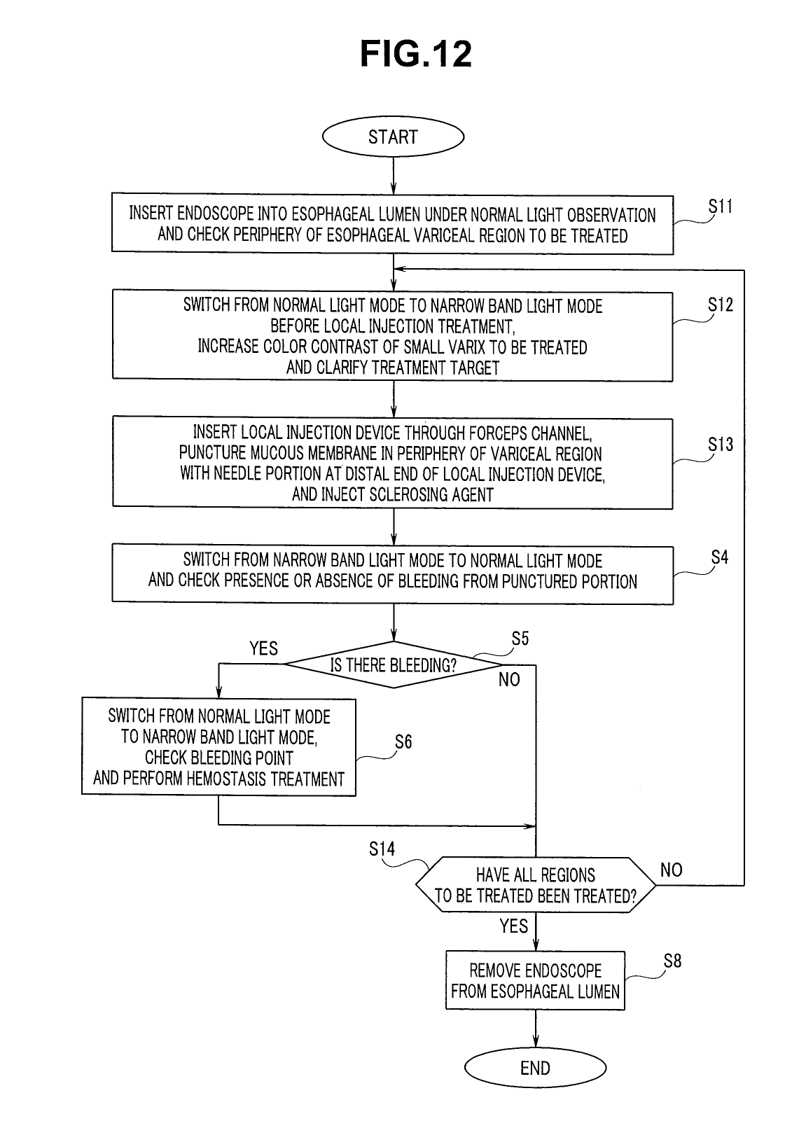

FIG. 12 is a flowchart illustrating a flow example of the method for endoscopic treatment of extravascular injection for an esophageal varix in EIS.

### [Checking Periphery of Esophageal Varix]

The operator inserts the endoscope 3 into the esophageal lumen under the normal light observation and checks the periphery of the esophageal variceal region to be treated (S11). First, the subject is irradiated with white light.

More specifically, as in the case of aforementioned S1, the operator causes the distal end portion of the insertion portion 3a to approach the vicinity of the esophageal varices and checks the periphery of the esophageal variceal region deflated by intravascular injection.

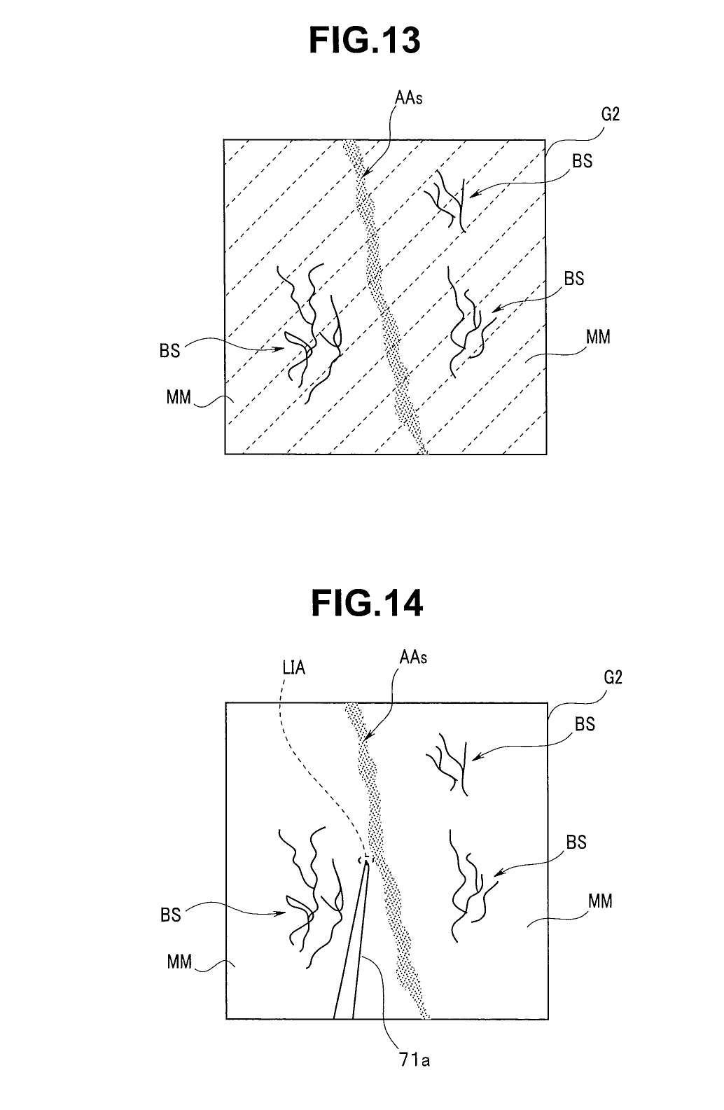

FIG. 13 is a diagram illustrating an example of an endoscopic image of the interior of the esophagus ES including an esophageal variceal region AAs deflated by intravascular injection and a periphery thereof. An endoscopic image G2 is displayed on the screen 5a of the observation monitor 5 in FIG. 13 and the endoscopic image G2 includes the esophageal variceal region AAs deflated by intravascular injection and two esophageal mucous membrane regions MM. Surface-layer blood vessels BS appear on some parts of the esophageal mucous membrane regions MM.

Since the endoscopic image G2 in FIG. 13 is an endoscopic image under normal light observation, the esophageal mucous membranes MM, the esophageal variceal region AAs deflated by intravascular injection and the surface-layer blood vessels BS are all displayed in red, and the deflated esophageal variceal region AAs has low contrast with respect to the surrounding esophageal mucous membranes MM. In FIG. 13, dotted diagonal lines are placed over the entire endoscopic image G2, indicating that the deflated esophageal variceal region AAs has low contrast with respect to the surrounding esophageal mucous membranes MM.

### [Switching to Narrow Band Light Observation Mode]

Returning to FIG. 12, the operator switches the observation mode of the endoscope apparatus 1 from the normal light observation mode to the narrow band light observation mode before local injection treatment on the periphery of the esophageal variceal region AAs, increases the color contrast of the deflated esophageal varix AAs and clarifies the treatment target (S12). That is, after irradiation with white light, switching is made to irradiation of a living tissue of the subject with narrow band light having a predetermined peak wavelength.

### [Local Injection]

Next, the operator inserts a local injection device for locally injecting a sclerosing agent into a mucous membrane in the periphery of the deflated esophageal variceal region AAs, through the forceps channel, punctures the mucous membrane in the periphery of the deflated esophageal varix with the needle portion at the distal end of the local injection device, and injects the sclerosing agent into the mucous membrane in the periphery of the deflated esophageal variceal region AAs (S13). That is, medicine is administered to the subject after irradiation with the narrow band light.

As shown in aforementioned FIG. 8, the mucous membrane in the periphery of the esophageal variceal region AAs is punctured with the needle portion 71a of the local injection device 71 from the distal end portion of the insertion portion 3a of the endoscope 3.

FIG. 14 is a diagram illustrating an example of an endoscopic image of the interior of the esophagus ES including the mucous membrane in the periphery of the deflated esophageal variceal region AAs.

Since FIG. 14 is an endoscopic image under narrow band light observation, the esophageal mucous membranes MM are displayed in yellow, the esophageal variceal region AAs is displayed in dark red and the surface-layer blood vessels BS are displayed in orange, the esophageal variceal region AAs is displayed with higher contrast with respect to the surrounding esophageal mucous membranes MM. As shown in FIG. 14, the needle portion 71a of the local injection device 71 is punctured at a local injection position LIA.

The sclerosing agent is, for example, Aethoxysklerol. After the local injection, venules in the periphery of the esophageal variceal region AAs are caused by the sclerosing agent to solidify and disappear. It is difficult to determine results of the treatment of the varices in real time in the normal light observation mode.

### [Checking Presence or Absence of Bleeding]

Next, the operator switches the observation mode from the narrow band light observation mode to the normal light observation mode and checks the presence or absence of bleeding from the punctured portion (S4). Note that S4 to S6 are the same as S4 to S6 in the processing in aforementioned FIG. 5.

When bleeding is detected (S5: YES) as a result of the determination of the presence or absence of bleeding (S5), the operator performs hemostasis treatment (S6).

### [Hemostasis Treatment]

The operator switches the observation mode from the normal light observation mode to the narrow band light observation mode, checks the bleeding point and performs hemostasis treatment (S6).

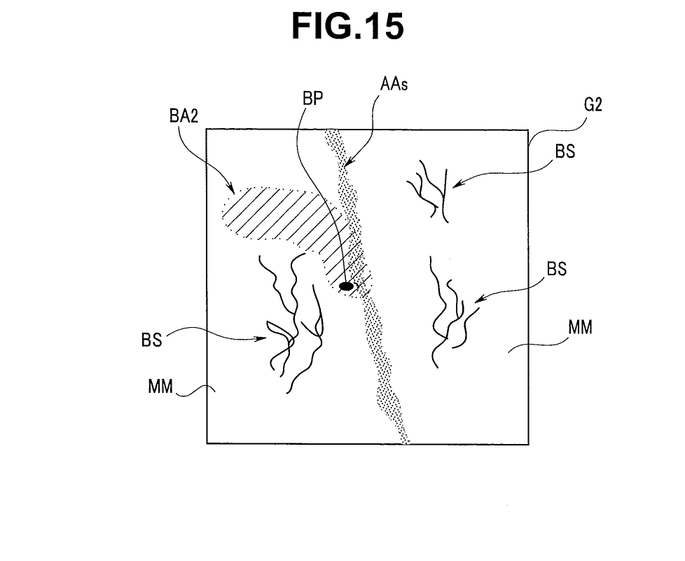

FIG. 15 is a diagram illustrating a case where bleeding has occurred during local injection. After detecting bleeding, the operator switches the observation mode to the narrow band light observation mode, and can visually recognize the bleeding point on the observation monitor 5 in the narrow band light observation mode as shown in FIG. 15. In the narrow band light observation mode, since a bleeding region BA2 shown by diagonal lines is displayed in yellow to orange and the bleeding point BP is displayed in yellow, the operator can identify the bleeding point BP.

As a result, the operator removes the local injection device 71 from the forceps channel, and can perform astriction treatment using a balloon provided in the insertion portion 3a or effectively perform hemostasis treatment by inserting the high-frequency scalpel 81 or the hemostasis forceps into the forceps channel.

After the hemostasis treatment, the operator switches the observation mode from the narrow band light observation mode to the normal light observation mode, thereby switches irradiation with narrow band light to irradiation with white light and checks whether or not the hemostasis treatment has been completed.

Upon confirming that the hemostasis treatment has been completed, the operator determines whether or not all the regions to be treated have been treated (S14), and if there still remain regions to be treated (S14: NO), the operator returns to S12 and repeats the aforementioned treatments in S12 to S16 on the other regions.

When the treatments on all regions to be treated have been completed and there remain no more varices to be treated (S14: YES), the operator removes the endoscope 3 from the esophageal lumen (S8).

Therefore, according to the method for endoscopic treatment of the aforementioned embodiment, the operator can perform EIS on the esophageal varices appropriately and speedily, and when further bleeding occurs, the operator can check the bleeding point and perform hemostasis treatment.

Especially, since an endoscopic image of the esophagus may shake due to heart stroke, by highlighting and clearly displaying the esophageal varices in the narrow band light observation mode as described above, the operator can perform medicine administration treatment such as local injection more easily.

### Second Embodiment

Next, a second embodiment will be described. While the first embodiment relates to a method for endoscopic treatment for medicinal treatment on esophageal varices, the second embodiment relates to a method for endoscopic treatment for medicinal treatment on ulcerative colitis.

Since an endoscope apparatus used for the method for endoscopic treatment in the second embodiment is similar to the endoscope apparatus 1 described in the first embodiment, description of the configuration of the apparatus will be omitted and the method for endoscopic treatment of the second embodiment will be described. Hereinafter, medicinal treatment on ulcerative colitis will be mainly described.

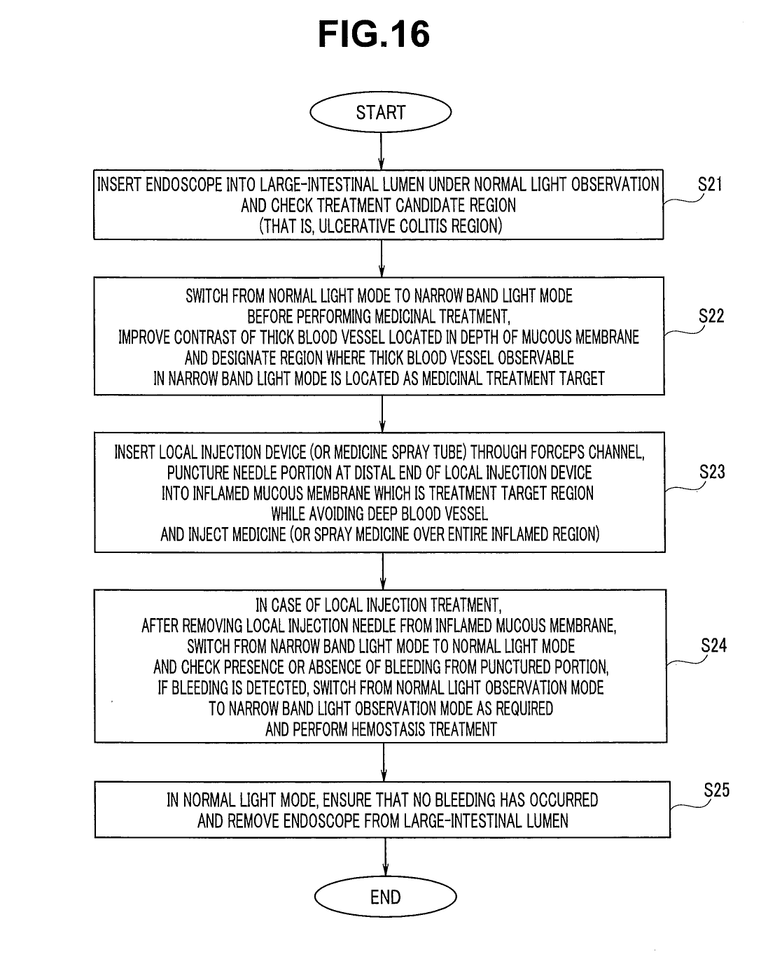

FIG. 16 is a flowchart illustrating a flow example of the method for endoscopic treatment of medicine administration for ulcerative colitis.

### [Checking Candidate Region to be Treated]

The operator inserts the endoscope 3 into the large-intestinal lumen of the large intestine under normal light observation and checks the ulcerative colitis region which is the candidate region to be treated (S21). That is, the subject is irradiated with white light to check the ulcerative colitis region.

More specifically, the operator switches the operating mode of the endoscope apparatus 1 to the normal light observation mode, inserts the insertion portion 3a from the anus of the subject, causes the distal end portion of the insertion portion 3a to approach the vicinity of the lesioned region while watching an image on the observation monitor 5 and operating the bending portion, and checks the region of the ulcerative colitis which is the candidate region to be treated.

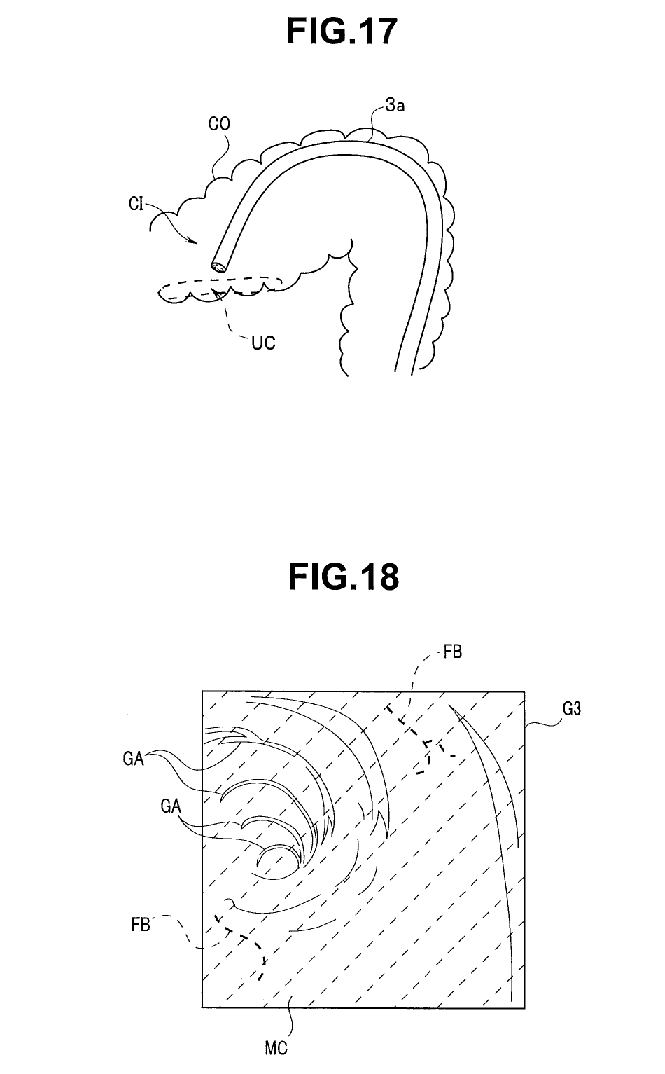

FIG. 17 and FIG. 18 are diagrams illustrating checking of the ulcerative colitis region. FIG. 17 is a diagram illustrating a condition in which the distal end portion of the insertion portion 3a of the endoscope 3 is brought close to the ulcerative colitis region (hereinafter, referred to as “UC region”) in the large-intestinal lumen CI of the large intestine CO and the UC region which is the inflamed region is included in a range of field of view of the endoscope 3.

When the operator causes the distal end portion of the insertion portion 3a of the endoscope 3 to approach the UC region, an endoscopic image of the interior of the large-intestinal lumen CI as shown in FIG. 18 is displayed on the observation monitor 5.

FIG. 18 is a diagram illustrating an example of an endoscopic image of the interior of the large-intestinal lumen CI including the UC region in the narrow band light observation mode. A plurality of creases GA formed on the inner wall of the large intestine CO and a large intestine mucous membrane region MC are displayed on the screen 5a of the observation monitor 5 as an endoscopic image G3. Thick blood vessels FB appear in the large intestine mucous membrane region MC.

Since FIG. 18 is an endoscopic image under normal light observation, both the large intestine mucous membrane region MC and the thick blood vessel FB are displayed in red. In FIG. 18, dotted diagonal lines are placed over the entire endoscopic image G3, the thick blood vessel in the depth of the mucous membrane has low contrast, making it difficult to view the image of the blood vessel below the inflamed region of the ulcerative colitis.

In FIG. 18, although no image of the blood vessel below the UC region which is the inflamed region is shown (that is, not visible), the image of the blood vessel below the inflamed region may be visible under normal light observation if it has a low to medium degree on the order of Matts grade 2 of endoscopic severity evaluation, but it is hardly visible.

### [Switching to Narrow Band Light Observation Mode]

Returning to FIG. 16, the operator switches the observation mode of the endoscope apparatus 1 from the normal light observation mode to the narrow band light observation mode before performing medicinal treatment on the large intestine mucous membrane region MC, improves the contrast of the thick blood vessel located in the depth of the mucous membrane and designates the region where the thick blood vessel which is observable in the narrow band light observation mode as the medicinal treatment target (S22). That is, after irradiation with white light, switching is made to irradiation of the living tissue of the subject with narrow band light having a predetermined peak wavelength.

This is because the observation mode is switched from the normal light observation mode to the narrow band light observation mode before the medicinal treatment, the relatively thick blood vessel in the depth of the mucous membrane can be displayed on the observation monitor 5 in a visually recognizable manner with high contrast.

Note that when the contrast of the thick blood vessel in the narrow band light observation mode is high, the region may be omitted from among the treatment targets and a region which is observable in the narrow band light observation mode and whose blood vessel has low contrast may be designated as the treatment target.

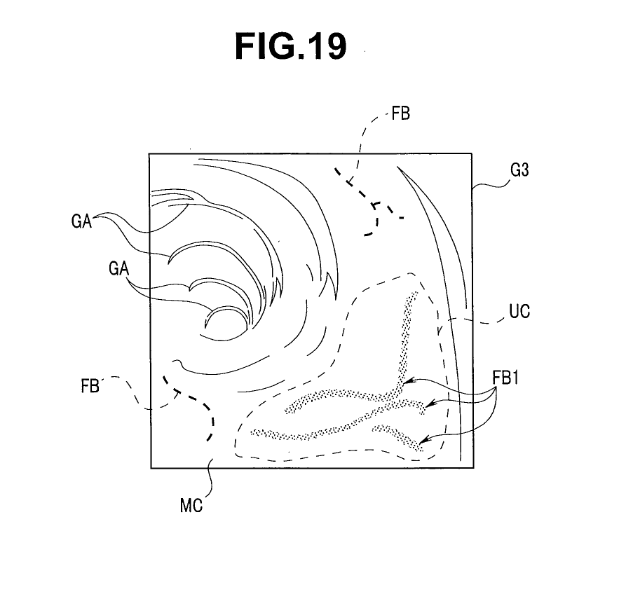

FIG. 19 is a diagram illustrating an example of an endoscopic image of the interior of the large-intestinal lumen CI including the UC region in the narrow band light observation mode. On the screen 5a of the observation monitor 5, a plurality of creases GA formed on the inner wall of the large intestine CO and thick blood vessels FB1 in the UC region in addition to the large intestine mucous membrane region MC are displayed as an endoscopic image G3. That is, the thick blood vessels FB1 in the UC region appear in the narrow band light observation mode.

Since FIG. 19 is an endoscopic image under narrow band light observation, the large intestine mucous membrane region MC is displayed in a yellow tone or reddish-brown tone. Moreover, the thick blood vessels FB1 in the UC region are hardly or not at all observable in the normal light observation mode, whereas the thick blood vessels FB1 in the UC region are displayed in red or green in the narrow band light observation mode.

### [Local Injection]

Next, the operator inserts the local injection device into the forceps channel of the endoscope 3, punctures the inflamed mucous membrane which is the treatment target region with the needle portion at the distal end of the local injection device while avoiding the deep blood vessel and injects medicine (S23). That is, medicine is administered to the subject after irradiation with narrow band light.

Note that medicine is injected into the inflamed mucous membrane using the local injection device here, but a medicine spray tube may be inserted through the forceps channel of the endoscope 3 to spray the medicine over the entire inflamed region.

As described above, medicine is administered to the inflamed mucous membrane of the subject by locally injecting the inflammation treatment medicine via a local injection needle or using a spray probe.

The medicine injected or sprayed is, for example, 5-ASA formulation such as Pentasa and Asacol.

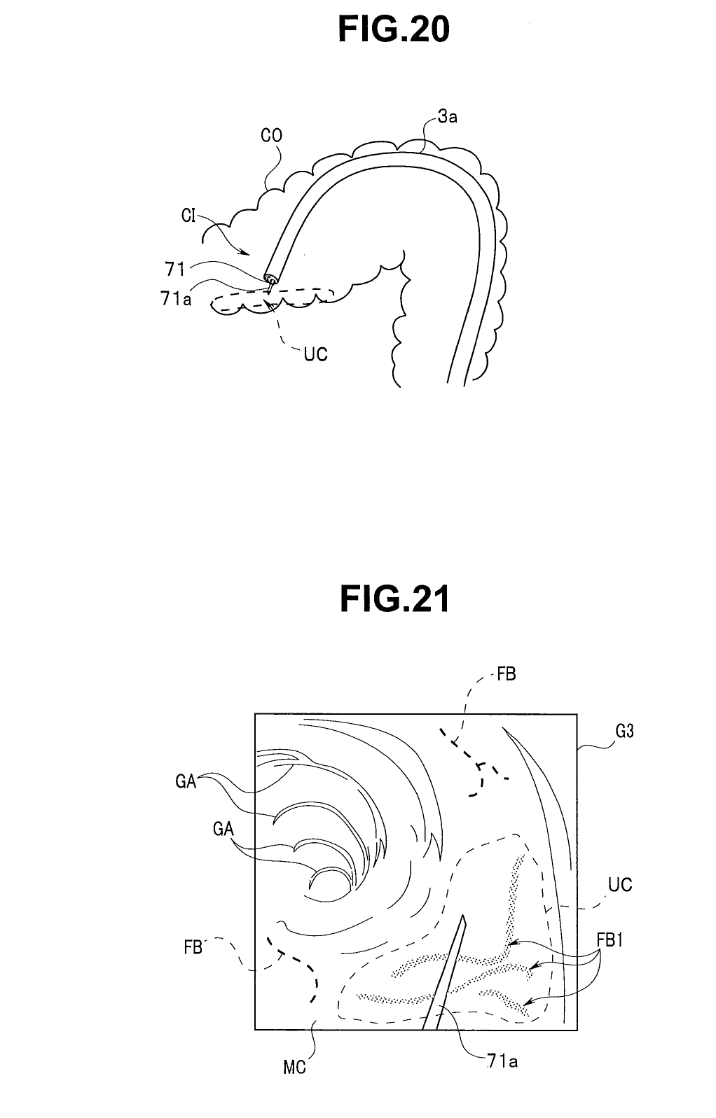

FIG. 20 and FIG. 21 are diagrams illustrating local injection into the UC region. FIG. 20 is a diagram illustrating a condition in which the needle portion 71a of the local injection device 71 from the distal end portion of the insertion portion 3a of the endoscope 3 is approaching the UC region to be treated. FIG. 21 is a diagram illustrating an example of an endoscopic image of the interior of the large-intestinal lumen CI including the UC region in the condition in FIG. 20.

Since FIG. 21 is an endoscopic image under narrow band light observation, the thick blood vessels FB1 in the UC region are displayed with high contrast with respect to the surrounding large intestine mucous membrane region MC.

### [Checking Presence or Absence of Bleeding and Hemostasis Treatment]

Next, in the case of local injection treatment, after removing the local injection needle 71a from the inflamed mucous membrane, the operator switches the observation mode from the narrow band light observation mode to the normal light observation mode and checks the presence or absence of bleeding from the punctured portion. When bleeding occurs, the operator switches the observation mode from the normal light observation mode to the narrow band light observation mode as required, checks the bleeding point and performs hemostasis treatment (S24).

After the hemostasis treatment, the operator switches the observation mode from the narrow band light observation mode to the normal light observation mode, thereby switches from irradiation with narrow band light to irradiation with white light, ensures that no bleeding occurs in the normal light observation mode and removes the endoscope 3 from the large intestine lumen (S25).

As described above, according to the method for endoscopic treatment of the aforementioned embodiment, the operator can perform medicine administration/treatment appropriately and speedily in treatment of ulcerative colitis, and when bleeding occurs, the operator can further perform hemostasis treatment after checking the bleeding point.

The aforementioned two embodiments are examples where the method for endoscopic treatment under narrow band light observation is applied to treatment of esophageal varix and treatment of ulcerative colitis, but the aforementioned method for endoscopic treatment is also applicable to medicinal treatment of other diseases such as interstitial cystitis.

For treatment of interstitial cystitis or the like, medicine is administered by locally injecting inflammation treatment medicine via a local injection needle or using a spray probe into the region of the interstitial cystitis of the subject.

Note that although the methods for endoscopic treatments according to the aforementioned two embodiments use a so-called frame-sequential endoscope apparatus using a monochrome image pickup device, a so-called simultaneous endoscope apparatus using a three primary color image pickup device or a complementary color image pickup device may also be used. In the case of a simultaneous endoscope apparatus, a plurality of light-emitting devices that emit their respective narrow band light beams may be used for an illumination apparatus and image acquiring timings may be controlled to prevent color mixing or whole wavelength information (reflected light) obtained from an object may be simultaneously detected.

Furthermore, according to the methods for endoscopic treatment according to the aforementioned two embodiments, the light source apparatus 4 uses a xenon lamp, but a light-emitting diode (LED) or laser diode (LD) may also be used to emit white light or band-limited light.

Furthermore, according to the methods for endoscopic treatments according to the aforementioned two embodiments, in the narrow band light observation mode, narrow band light having a predetermined peak wavelength is radiated onto a subject as band-limited light, but light including narrow band light having a predetermined peak wavelength and having a broad range or light including not only narrow band light having a predetermined peak wavelength but also wideband light in other wavelength bands may also be radiated onto the subject as band-limited light.

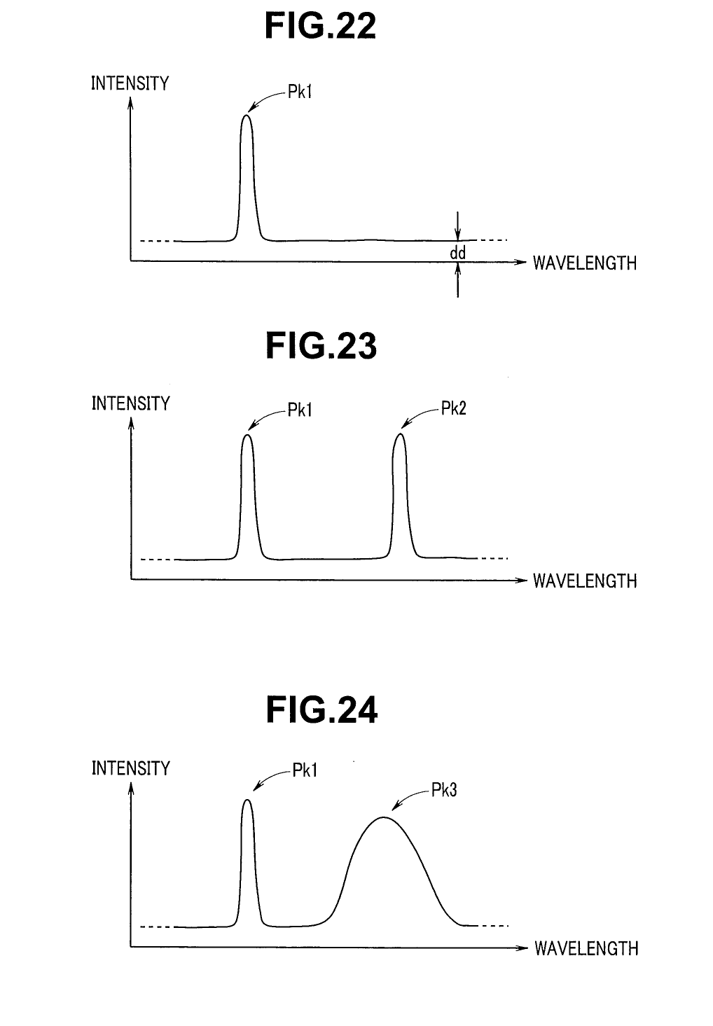

FIG. 22 to FIG. 24 are diagrams illustrating band-limited light. FIG. 22 is a diagram illustrating a relationship between wavelength and intensity of band-limited light including narrow band light having one predetermined peak wavelength and having a broad range. The band-limited light in FIG. 22 has a wavelength band including a peak Pk1, and has non-zero intensity dd in other wavelength bands.

FIG. 23 is a diagram illustrating a relationship between wavelength and intensity of band-limited light including narrow band light having two predetermined peak wavelengths and having a broad range. The band-limited light in FIG. 23 has a wavelength band including a peak Pk1 and a wavelength band including a peak Pk2 generated by a filter, and has non-zero intensity in other wavelength bands.

FIG. 24 is a diagram illustrating a relationship between wavelength and intensity of band-limited light including narrow band light having one predetermined peak wavelength and one wide band light. The band-limited light in FIG. 24 has a wavelength band including a peak Pk1 and wide band light including a peak Pk3, and has non-zero intensity in other wavelength bands. The light shown in FIG. 23 can be obtained by combining wide band light generated by fluorescent excitation light and narrow band light generated by a light-emitting diode (LED) or a laser diode (LD).

That is, not only narrow band light having a simple peak wavelength as described in the first and second embodiments but also the light described in FIG. 22 to FIG. 24 may be used as band-limited light for the methods for endoscopic treatment according to the aforementioned first and second embodiments.

The present invention is not limited to the aforementioned embodiments, but various modifications or changes or the like can be made without departing from the spirit and scope of the present invention.

What is claimed is:

1. A method for endoscopic treatment that performs treatment on a subject under an endoscope, the method comprising:
irradiating the subject with white light;
switching, after irradiation with the white light, to irradiation of a living tissue of the subject with band-limited light having a predetermined peak wavelength; and
administering medicine to the subject after irradiation with the band-limited light.

2. The method for endoscopic treatment according to claim 1, wherein the medicine is administered by locally injecting a sclerosing agent into varices of the subject via a local injection needle.

3. The method for endoscopic treatment according to claim 1, wherein the medicine is administered by locally injecting inflammation treatment medicine into an inflamed mucous membrane of the object via a local injection needle or spraying inflammation treatment medicine over an inflamed mucous membrane of the subject using a spray probe.

4. The method for endoscopic treatment according to claim 1, wherein the medicine is administered by locally injecting inflammation treatment medicine into an inflamed mucous membrane of the object via a local injection needle or spraying inflammation treatment medicine over a region of interstitial cystitis of the subject using a spray probe.

5. The method for endoscopic treatment according to claim 1, further comprising performing, after irradiation with the band-limited light, hemostasis treatment on a bleeding blood vessel of the living tissue using an electric scalpel or hemostasis forceps.

6. The method for endoscopic treatment according to claim 5, further comprising switching, after the hemostasis treatment, from irradiation with the band-limited light to irradiation with the white light.

7. The method for endoscopic treatment according to claim 1, wherein the band-limited light has a peak wavelength in spectral characteristics in a red band of a visible range between a wavelength band including a maximum value and a wavelength band including a minimum value in hemoglobin light absorption characteristics of the living tissue of the subject.

8. The method for endoscopic treatment according to claim 7, wherein in radiation of the band-limited light, light including narrow band light of 585 nm to 630 nm is radiated.
